(19) United States
(12) Patent Application Publication
(10) Pub. No.: US 2026/0272424 A1
(43) Pub. Date: Sep. 17, 2026

(54) ULTRASOUND DIAGNOSTIC SYSTEM

(71) Applicant: FUJIFILM Corporation, Tokyo (JP)

(72) Inventor: Natsumi DANNO, Tokyo (JP)

(73) Assignee: FUJIFILM Corporation, Tokyo (JP)

(21) Appl. No.: 19/558,372

(22) Filed: Mar. 5, 2026

(30) Foreign Application Priority Data
Mar. 11, 2025 (JP) 2025-038733

(51) Int. Cl. A61B 8/00; A61B 8/06; G06T 7/00; G16H 15/00

(52) U.S. Cl. CPC A61B 8/5223; A61B 8/06; G06T 7/0012; G16H 15/00; G06T 2207/10132; G06T 2207/20081; G06T 2207/30096; G06T 2207/30101

(57) ABSTRACT

An ultrasound diagnostic system comprises a processor configured to execute determination processing and output suggestion information. The determination processing determines, from ultrasound images such as a vascular image or a blood flow waveform obtained in a current examination, an imaging scene and a lesion state represented by the ultrasound images. The determination processing is constructed by machine learning using, for example, an ultrasound image as input and information on an imaging scene and a lesion state corresponding to the ultrasound image as labeled data. The suggestion information suggests an additional examination or measurement based on a combination of the imaging scene and the lesion state determined by the determination processing. The suggestion information is generated, for example, by referring to a look-up table in which the combination of the imaging scene and the lesion state and the additional examination or measurement corresponding to the combination are registered.

### CROSS REFERENCE TO RELATED APPLICATION

This application claims priority to Japanese Patent Application No. 2025-038733 filed on Mar. 11, 2025, which is incorporated herein by reference in its entirety including the specification, claims, drawings, and abstract.

### BACKGROUND OF THE DISCLOSURE

### Field of the Disclosure

The present disclosure relates to an ultrasound diagnostic system.

### Description of the Related Art

In an ultrasound examination, a series of steps are generally sequentially performed according to a procedure determined in an examination guideline or the like. For example, in an ultrasound examination of abdomen, in each step, a predetermined cross section of an organ to be diagnosed is displayed as a tomographic image. Required measurement is performed on each tomographic image. At the end of each step, a step execution result (tomographic image, measurement result, findings, and the like) is stored.

In order to support an examiner in execution process of the series of steps, an ultrasound diagnostic apparatus has several examination protocols as an examination assist function. For example, in a case where a specific examination protocol is selected and then executed, execution conditions set in advance for each step are automatically set. In addition, during the execution of the step, the required measurement is started. In a case where a store operation is performed, the process automatically transitions to the next step. Each step constituting the examination protocol is also referred to as a view from the viewpoint of image observation.

In recent years, with the development of artificial intelligence technology such as a neural network, the use of artificial intelligence technology has also been advanced in an ultrasound diagnostic apparatus.

In the device disclosed in JP2021-164533A, the acquisition unit acquires first Doppler waveform data related to the left ventricular inflow blood flow. The detection unit detects the positions of the E-wave and the A-wave in the first Doppler waveform data using a trained model that has been trained using training data including at least the positions of the E-wave and the A-wave in each of a plurality of pieces of second Doppler waveform data related to a left ventricular inflow blood flow and the plurality of pieces of second Doppler waveform data, and the first Doppler waveform data.

The apparatus disclosed in JP6547612B obtains several feature amounts from a blood flow image representing a blood flow and a tomographic image representing a tissue, and determines whether or not each blood flow region is a neovascularization using the feature amounts by a machine learning-based evaluation unit.

The device disclosed in JP2023-509514A generates a plaque-based disease evaluation from a raw medical image by identifying features of a plaque, a blood vessel, and the like by using artificial intelligence or a machine learning imaging analysis algorithm and obtaining a quantified plaque parameter.

In a case where an examiner finds a possibility of an abnormality from various ultrasound images obtained in a step in an examination protocol being executed, the examiner may execute additional measurement for the images or additional ultrasound examination for related portions to examine the abnormality in more detail. However, there is a possibility that an error may occur due to fatigue of the examiner, lack of experience, or the like. There is a demand for support from a system in order to reduce such an error.

### SUMMARY OF THE DISCLOSURE

An ultrasound diagnostic system according to an aspect of the present disclosure includes a processor, in which the processor is configured to: execute, from at least one of a vascular image, a blood flow waveform, or an envelope of the blood flow waveform, obtained by transmission and reception of ultrasonic waves to and from a subject in an ultrasound examination step being executed, determination processing of determining an imaging scene and a lesion state of the ultrasound examination step being executed; and output, based on a combination of the determined imaging scene and lesion state, suggestion information for suggesting additional ultrasound examination or measurement for the ultrasound examination step being executed.

In a certain aspect, the processor is configured to determine the imaging scene and the lesion state in the ultrasound examination step being executed, by using a machine learning model trained to output, in a case where an input of at least one of the vascular image, the blood flow waveform, or an image of the envelope is received, the imaging scene and the lesion state corresponding to the input.

In another aspect, the processor is further configured to present a finding candidate for the subject, based on a result obtained by executing an ultrasound examination or measurement specified by a user among the additional ultrasound examination or measurement shown in the suggestion information and on the imaging scene and the lesion state determined by the determination processing.

Here, the processor may be further configured to create a report document including a finding created based on the finding candidate.

In still another aspect, the processor is further configured to execute determination of appropriateness for an approach of an ultrasound probe from at least one of the vascular image, the blood flow waveform, or an image of the envelope, and the determination of the appropriateness for the approach is performed using a second machine learning model trained to output, in a case where an input of at least one of the vascular image, the blood flow waveform, or the image of the envelope is received, the appropriateness of the approach corresponding to the input.

### BRIEF DESCRIPTION OF THE DRAWINGS

FIG. 1 is a diagram showing an example of a functional configuration of an ultrasound diagnostic system of an embodiment.

FIG. 2 is a diagram showing an example of an internal configuration of determination processing.

FIG. 3 is a diagram showing an example of an LUT constituting imaging scene determination.

FIG. 4 is a diagram showing an example in which a blood vessel name is displayed on a B-mode tomographic image.

FIG. 5 is a diagram showing an example of a LUT constituting additional examination suggestion processing.

FIG. 6 is a diagram schematically showing a configuration of a screen provided by the ultrasound diagnostic system.

FIG. 7 is a diagram schematically showing a configuration of a screen in a case where an abnormality is detected from an ultrasound image.

FIG. 8 is a diagram schematically showing a configuration of a screen for inputting an instruction for an additional examination or the like in a case where an abnormality is detected by an ultrasound examination.

FIG. 9 is a diagram schematically showing a configuration of a screen in a case where an instruction to execute an additional examination protocol is given.

FIG. 10 is a diagram showing a template of a report generated by the ultrasound diagnostic system.

FIG. 11 is a diagram showing an example of a report in a case where normal is determined by the ultrasound examination.

FIG. 12 is a diagram showing an example of a report in a case where a plaque is found by the ultrasound examination.

### DESCRIPTION OF THE PREFERRED EMBODIMENTS

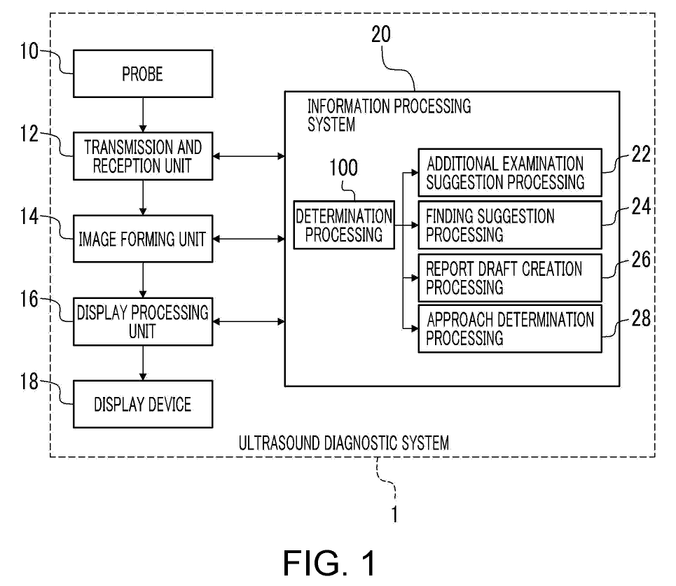

FIG. 1 shows an ultrasound diagnostic system 1 according to the embodiment. The ultrasound diagnostic system 1 is a medical system that is installed in a medical institution and is used in an ultrasound examination. The ultrasound diagnostic system 1 comprises a probe 10, a transmission and reception unit 12, an image forming unit 14, a display processing unit 16, a display device 18, and an information processing system 20 as functional elements.

The probe 10 is an ultrasound probe that transmits and receives ultrasonic waves. A vibration element array consisting of a plurality of vibration elements is provided in the probe 10. An ultrasound beam is formed by the vibration element array, and the ultrasound beam is electronically scanned. As a result, a beam scanning surface is formed.

The transmission and reception unit 12 supplies a plurality of transmission signals in parallel to the vibration element array at the time of transmission, and applies phasing addition (delay addition) to a plurality of reception signals from the vibration element array at the time of reception. As a result, the beam data is configured. One reception frame data is generated by scanning with one ultrasound beam. One piece of the reception frame data is composed of a plurality of pieces of beam data arranged in an electronic scanning direction. Each piece of the beam data is composed of a plurality of pieces of echo data arranged in a depth direction.

The reception frame data sequentially output from the transmission and reception unit 12 is sequentially input to the image forming unit 14. The image forming unit 14 includes a digital scan converter (DSC). The DSC generates the display frame data sequence from the reception frame data sequence. The DSC has a coordinate transformation function, a pixel interpolation function, and the like. One display frame data constitutes one tomographic image data. Although not shown, an image forming unit that forms various ultrasound images is provided in FIG. 1. The various ultrasound images include, for example, two-dimensional tomographic images such as a B-mode tomographic image, a color Doppler image (also referred to as a color flow image), and a power Doppler image. In addition, the formed ultrasound image may include an image configured by combining a plurality of types of two-dimensional tomographic images, such as an image in which a color Doppler image or a power Doppler image is superimposed on a B-mode tomographic image. In addition, an image (for example, a signal waveform or a temporal change in intensity) in a form other than the two-dimensional tomographic image, such as a blood flow waveform image generated by the pulse Doppler mode (also referred to as a PW mode; PW is an abbreviation of Pulse Wave), is also an example of the ultrasound image formed by the image forming unit 14.

The display processing unit 16 has an image combining function, a color processing function, and the like. A display screen displayed on the display device 18 is formed by the display processing unit 16. First, the ultrasound image is included in the display screen. The ultrasound image is a video image in a case of a real-time operation and is a still image in a case of a freeze state. The display device 18 is configured by an LCD, an organic EL display, or the like. The display screen includes various images generated in the information processing system 20. Among them, a step list image, a measurement list image, a measured value graph, an examination value graph, and the like can be included. The step list image shows a list of examination steps included in the examination protocol being executed.

The information processing system 20 executes information processing such as control of the operation of each component of the ultrasound diagnostic system 1, various types of analysis processing on the ultrasound image, generation of various images to be displayed on the display screen, and a layout configuration of the display screen. The information processing system 20 comprises, for example, a processor such as a central processing unit (CPU) or a memory such as a random access memory (RAM) as hardware.

The information processing executed by the information processing system 20 includes the determination processing 100, the additional examination suggestion processing 22, the finding suggestion processing 24, the report draft creation processing 26, and the approach determination processing 28.

The determination processing 100 is processing of determining an imaging scene, blood vessel identification, and a lesion state. The determination processing 100 described here is an example in a case where a circulatory system examination such as a carotid artery is targeted. These determinations are performed based on various ultrasound images generated by the image forming unit 14, data (for example, a measurement value such as a diameter of a blood vessel or a maximum blood flow velocity) obtained based on the ultrasound images, and the like. These determination results are used by the additional examination suggestion processing 22, the finding suggestion processing 24, the report draft creation processing 26, and the approach determination processing 28.

The additional examination suggestion processing 22 is processing of suggesting an examination or measurement to be additionally performed for the currently executed examination based on the imaging scene and the lesion state determined by the determination processing 100.

The finding suggestion processing 24 is processing of suggesting the content of the finding to be registered in a predetermined patient database as one of the items of the examination result.

The finding is a wording indicating the content of the abnormality determined or estimated by the examination, and includes, for example, a wording or numerical information describing a symptom name or a degree thereof, and a description describing a site where the symptom is seen. The findings are entered by a doctor, a laboratory technician, or the like. The finding suggestion processing 24 suggests a wording that is a candidate for a finding entered by a doctor or the like, and creates the finding candidate based on the imaging scene and the lesion state determined by the determination processing 100. A doctor or the like confirms the content of the finding candidate, confirms the wording of the finding after making a correction as necessary, and registers the finding in the patient database.

The report draft creation processing 26 is processing of creating a draft of a report of the examination result. The report includes patient information (for example, a patient ID, a name, a gender, a medical history, and the like) and an examination result. The examination result includes one or more of findings, measurement results, images as evidence, and the like, which are entered by a doctor or the like. The report draft creation processing 26 creates a draft of the report based on the imaging scene and the lesion state determined by the determination processing 100. The content of the findings in this draft may be borrowed from the finding candidate generated by the finding suggestion processing 24 or the final version of the findings confirmed and corrected by the doctor or the like.

The approach determination processing 28 is processing of determining the appropriateness of the approach of the probe 10 to the subject (that is, the body of the patient or the examinee). Here, the approach of the probe 10 is a method of applying the probe 10 for imaging the cross section of the observation target, and is defined by a position and an orientation at which the probe 10 is applied, a degree of pushing of the probe 10, a tilt (angle) of the probe 10, and the like. The position and orientation of the probe 10 can be determined from a tomographic image such as a B-mode image or a color Doppler image. For example, it is possible to determine whether or not the cross section suitable for the examination appears in the tomographic image, whether or not the color representing the blood flow velocity appearing in the color Doppler image is appropriate, and the like. In addition, the degree of pushing of the probe 10 (for example, the degree of pushing strength) can be determined from the degree of deformation of the blood vessel appearing in the tomographic image.

Next, the details of the determination processing 100 will be described. As described above, in the determination processing 100, the imaging scene, the blood vessel identification, and the lesion state are determined.

Here, the imaging scene is a scene of observing an organ (for example, an organ or a blood vessel) for diagnosing a lesion. The imaging scene defines one or more of a structure such as an organ, a specific position or shape in the structure, an examination condition (for example, a mode in ultrasound diagnosis), a cardiac phase, and the like. The imaging scene is often defined by, for example, a diagnostic guideline, a hospital, or a facility.

Imaging scenes such as a vascular short-axis cross section of a common carotid artery stenosis part, an internal carotid artery (that is, ICA) blood vessel branch part, and an IMT-C10 cross section are well known. ICA is an abbreviation for internal carotid artery. In addition, IMT-C10 is the IMT (that is, the intima-media thickness) at the distal wall site 10 mm from the transition portion between the common carotid artery and the carotid sinus on the central side.

The IMT-C10 cross section is an imaging scene that is often used to check the risk of developing a cardiovascular event and the like, and is a scene in which a longitudinal cross section of the carotid artery of the above-described site is observed in the B-mode. In the diagnosis, the thickness of the arterial wall of the site is measured from the B-mode tomographic image at this time.

In addition, the blood vessel identification is processing of identifying individual blood vessels appearing in the ultrasound image (that is, determining identification names of the respective blood vessels). For example, in the examination of the IMT-C10 cross section, the common carotid artery is the main observation target, but in the ultrasound field image, in addition to the common carotid artery, the carotid sinus and the internal carotid artery also appear. In the blood vessel identification, not only the blood vessel of the main observation target but also the identification name of the other blood vessel is determined.

In addition, the lesion state is a state of a lesion occurring in an organ. The lesion state is defined by one or more of the type, degree, and property of the lesion.

For example, there are types of lesions in the circulatory system, such as plaque, stenosis, obstruction, arterial dissection, arteriosclerosis, vasculitis, aneurysm, varicose vein, sinus bradycardia, atrial fibrillation, and the like. In addition, for example, for each type of lesion, several classifications are often provided for the degree or properties of the lesion for diagnosis. For example, for a certain type of lesion, several degrees of severity are defined. In addition, for a certain type of lesion, several classifications are defined for the properties thereof.

For example, the state of the plaque of the carotid artery is evaluated from several viewpoints such as echo brightness, uniformity, surface properties, and mobility. For example, there are classifications of high brightness type, equal brightness type, and low brightness type for echo brightness, and classifications of uniform type and non-uniform type for uniformity. Such brightness classification, uniformity classification, or a combination of both is an element that defines the properties of the plaque. In addition, the surface properties are classified into smooth, irregular, ulcer, and the like, and this classification is also one element of the properties of the plaque. In addition, the presence or absence of mobility is also one of the important properties of the plaque. In a case where the plaque shows mobility, there is a high risk of embolism or the like. In addition, the thickness of the plaque is a numerical indicator indicating the degree of the plaque. In addition, the size of the range of the plaque in the vascular long-axis direction, the area or the occupancy of the plaque in the vascular cross section, and the like may be used as a numerical indicator indicating the degree of the plaque.

In addition, in the case of the arterial dissection, characteristic structures such as an intramural hematoma and a double lumen are observed in the B-mode tomographic image, and characteristic blood flow patterns such as a helical blood flow and a false lumen blood flow are observed in the blood flow image obtained by the color Doppler or the like. These characteristic structures and blood flow patterns are elements that define the properties of the arterial dissection lesion.

In addition, the aneurysm is mainly seen in the common carotid artery, the internal carotid artery origin, the subclavian artery, and the like, and is basically vasodilation due to arteriosclerosis, but may be complicated with Takayasu arteritis. The aneurysm has a fusiform shape, a sac shape, and the like as a classification of a shape property. In addition, the presence or absence of a thrombus inside the aneurysm and the presence or absence of mobility are also important elements of the properties of the aneurysm.

In one example, the lesion state is a type of lesion. In another example, the lesion state is an index value (for example, severity) indicating a degree of a lesion. In still another example, the lesion state may be treated as an index value indicating a degree of a lesion as the lesion state. In addition, the characteristics of the lesion may be used as the lesion state. In still another example, the lesion state may be defined by a combination of two or more of the type, degree, and property of the lesion.

For example, in a case where a plaque is found from an ultrasound image of the common carotid artery, the lesion state of the common carotid artery may be expressed as “plaque present”. This is an example in a case where the type of lesion is treated as the lesion state.

In addition, in a case where it is found from the ultrasound image that the plaque is a high-brightness non-uniform type, the lesion state of the common carotid artery may be expressed as "plaque of high-brightness non-uniform type is present". This is an example in which the lesion state is represented by a combination of the type and the properties of the lesion. In a case where the thickness of the plaque is X millimeters, the lesion state may be expressed as "plaque with high brightness non-uniform type with a thickness of X millimeters".

In addition, a combination of the type and the severity of the lesion may be used as the lesion state.

In addition, a numerical value determined as an indicator of the atherosclerosis, such as the intima-media thickness (IMT), the stenosis rate, or the arterial diameter in the arterial contraction phase, or a combination thereof may be used as the lesion state of the artery of interest.

The imaging scene, the blood vessel identification, and the lesion state have been described above. In the determination processing 100 of the present embodiment, the imaging scene, the blood vessel identification, and the lesion state are determined using a machine learning model.

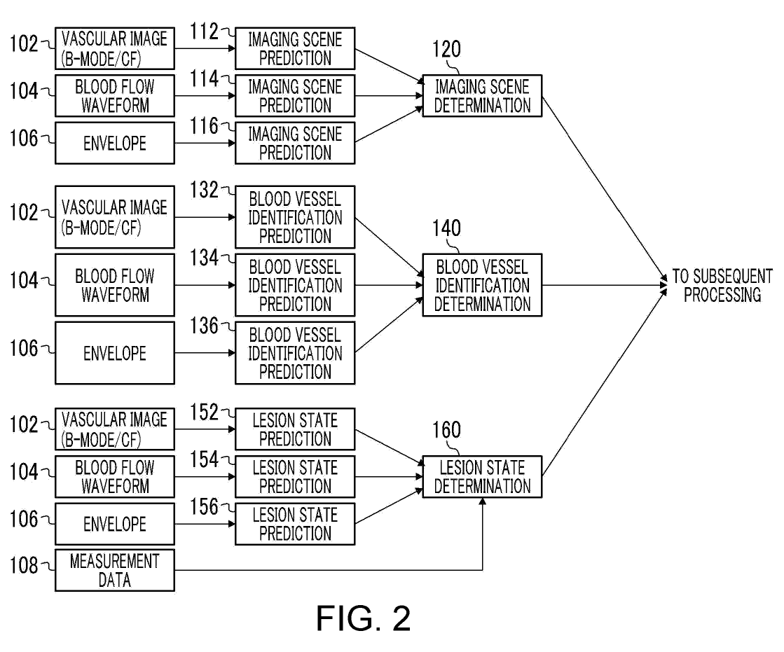

FIG. 2 shows an example of a detailed configuration of the determination processing 100. In this example, the determination is performed using the vascular image 102, the blood flow waveform 104, the envelope 106, and the like obtained by the ultrasound examination. The vascular image 102 is a tomographic image of the ultrasound image formed by the image forming unit 14. That is, the vascular image 102 is a B-mode tomographic image, a color Doppler image, a combination of a color Doppler image and a B-mode tomographic image, or the like. Here, since the examination of the circulatory system such as the carotid artery is taken as an example for the description, the blood vessel to be observed is displayed in the tomographic image. The blood flow waveform 104 is an image representing a waveform of the blood flow in the ultrasound image formed by the image forming unit 14. For example, the blood flow waveform 104 is the Doppler waveform data acquired in the pulse Doppler mode or the continuous wave Doppler mode. The envelope 106 is time-series data obtained by connecting envelopes of the blood flow waveform 104, that is, maximum values of the waveform 104 at each time point.

In the example of FIG. 2, the determination processing 100 has nine machine learning-based prediction models of the imaging scene predictions 112, 114, and 116, the blood vessel identification predictions 132, 134, and 136, and the lesion state predictions 152, 154, and 156.

The imaging scene prediction 112 is a model that predicts an imaging scene from the vascular image 102. In a case where the vascular image 102 is input, the imaging scene prediction 112 outputs identification information (for example, an identification name representing the imaging scene) of the imaging scene represented by the vascular image 102. In addition, the imaging scene prediction 112 may output, in addition to the identification information of the imaging scene predicted from the vascular image 102, a degree of certainty of the prediction (that is, a value corresponding to a probability that the prediction is correct). The imaging scene prediction 112 may output a plurality of prediction results (that is, identification information of the imaging scene) having a high degree of certainty for the input one vascular image 102.

The imaging scene prediction 112 is constructed by, for example, machine learning. The training data used in the machine learning includes a set of a vascular image and labeled data. In this case, the labeled data is identification information of an imaging scene represented by the vascular image. For example, a large amount of training data such as a set of the vascular image of the IMT-C10 cross section and the imaging scene name “IMT-C10 cross section” and a set of the vascular image of the short-axis cross section of the vertebral artery and the imaging scene name “vertebral artery, short axis” is prepared, and the imaging scene prediction 112 is constructed by causing the machine learning model to learn the training data.

The imaging scene prediction 114 is a model that predicts an imaging scene from the blood flow waveform 104. In a case where the blood flow waveform 104 is input, the imaging scene prediction 114 outputs identification information of the imaging scene represented by the blood flow waveform 104. In addition, the imaging scene prediction 112 may output the degree of certainty of the prediction in addition to the identification information of the imaging scene predicted from the vascular image 102. The imaging scene prediction 112 may output a plurality of prediction results (that is, identification information of the imaging scene) having a high degree of certainty for the input one vascular image 102.

The imaging scene prediction 114 is constructed by, for example, machine learning. The training data used in the machine learning includes a set of a blood flow waveform and labeled data. In this case, the labeled data is identification information of an imaging scene represented by the blood flow waveform. For example, a large amount of training data such as a combination of a blood flow waveform obtained in a case where the IMT-C10 cross section is examined and an imaging scene name "IMT-C10 cross section" is prepared, and the imaging scene prediction 114 is constructed by training the machine learning model with the training data.

The imaging scene prediction 116 is a model that predicts an imaging scene from the envelope 106. In a case where the envelope 106 is input, the imaging scene prediction 116 outputs identification information of the imaging scene represented by the envelope 106. In addition, the imaging scene prediction 116 may output the degree of certainty of the prediction in addition to the identification information of the imaging scene predicted from the envelope 106. The imaging scene prediction 116 may output a plurality of prediction results (that is, identification information of the imaging scene) having a high degree of certainty for the input one envelope 106.

The imaging scene prediction 116 is constructed by, for example, machine learning. The training data used in the machine learning includes a set of the envelope and the labeled data. In this case, the labeled data is identification information of the imaging scene represented by the envelope. For example, a large amount of training data such as a combination of an envelope obtained in a case where the IMT-C10 cross section is examined and an imaging scene name "IMT-C10 cross section" is prepared, and the imaging scene prediction 116 is constructed by causing the machine learning model to learn the training data.

Preprocessing such as noise removal and brightness correction is performed on images such as a vascular image, a blood flow waveform, and an envelope used for training the imaging scene predictions 112, 114, and 116.

The blood vessel identification predictions 132, 134, and 136 are models that perform blood vessel identification prediction from the vascular image 102, the blood flow waveform 104, and the envelope 106. The blood vessel identification predictions 132, 134, and 136 are constructed by machine learning using a large amount of training data, similarly to the imaging scene predictions 112, 114, and 116. The labeled data used for training the blood vessel identification predictions 132, 134, and 136 includes a blood vessel name represented by the corresponding image (vascular image, blood flow waveform, or envelope). In addition, the labeled data may include information indicating whether the corresponding image is obtained by imaging a short-axis cross section or a long-axis cross section of the blood vessel, in addition to the blood vessel name.

The lesion state predictions 152, 154, and 156 are models that predict the lesion state. The lesion state predictions 152, 154, and 156 are constructed by machine learning using a large amount of training data, similarly to the imaging scene predictions 112, 114, and 116. The labeled data in the training data used for training the lesion state predictions 152, 154, and 156 includes one or more of information indicating a lesion state specified from the corresponding image, for example, a type name indicating a type of a lesion, a numerical value indicating a degree of a lesion, or a property name indicating a property of a lesion. In addition, the training data used for the learning of the lesion state predictions 152, 154, and 156 includes data for a normal (that is, no lesion) subject. The labeled data in the training data for the normal subject includes information indicating that the subject is “normal”.

The machine learning model used in each prediction model described above is, for example, a neural network model using a deep learning technique. However, the present disclosure is not limited thereto, and other machine learning models may be used.

The imaging scene determination 120 determines the imaging scene by integrating the prediction results of the three imaging scene predictions 112, 114, and 116. The imaging scene determination 120 may be implemented as, for example, a look-up table (LUT).

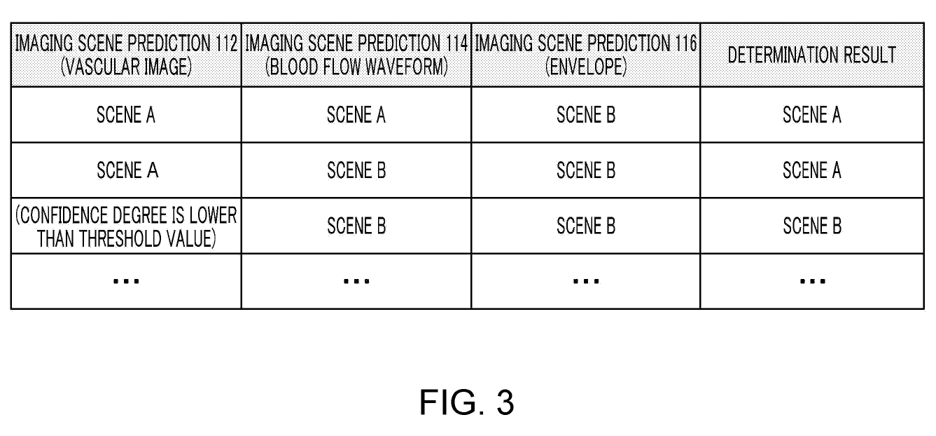

FIG. 3 shows an example of such a LUT. In the LUT illustrated in FIG. 3, the determination result of the imaging scene determination 120 corresponding to each combination of the prediction results of the imaging scene predictions 112, 114, and 116 is registered. The case where the three prediction results match is obvious and thus is not shown in the LUT. The third row of the LUT is an example in which the prediction result of the imaging scene prediction 112 based on the vascular image 102 is considered invalid because the degree of certainty of the prediction result is low (that is, less than a predetermined threshold value), and the determination is performed from the prediction results of the imaging scene predictions 114 and 116.

The implementation method of the imaging scene determination 120 is not limited to the LUT. Instead of this, for example, the imaging scene determination 120 may be implemented as a machine learning model.

In addition, a set of three imaging scene predictions 112, 114, and 116 and the imaging scene determination 120 may be replaced with one machine learning model. In this case, the machine learning model is a model that receives the vascular image 102, the blood flow waveform 104, and the envelope 106 as inputs and outputs a determination result of the imaging scene.

The blood vessel identification determination 140 performs the identification of the blood vessel by integrating the prediction results of the three blood vessel identification predictions 132, 134, and 136. The lesion state determination 160 determines the lesion state by combining the prediction results of the three lesion state predictions 152, 154, and 156, and the measurement data obtained by the measurement (for example, the measurement of the blood vessel diameter, the maximum blood flow velocity, and the like) performed on the ultrasound image. The implementation of the blood vessel identification determination 140 and the lesion state determination 160 can be performed by various implementation methods such as the LUT or the machine learning model, as in the case of the imaging scene determination 120. In addition, a set of the three blood vessel identification predictions 132, 134, and 136 and the blood vessel identification determination 140 may be replaced with one machine learning model, and similarly, a set of the three lesion state predictions 152, 154, and 156 and the lesion state determination 160 may be replaced with one machine learning model.

In some examinations, one or two of the vascular image 102, the blood flow waveform 104, and the envelope 106 may not be obtained. In such a case, the determination processing 100 performs the determination of the imaging scene or the like using the image obtained in the examination among the vascular image 102, the blood flow waveform 104, and the envelope 106.

In some examination steps, one or two of the vascular image 102, the blood flow waveform 104, and the envelope 106 may not be required. In such a case, the determination processing 100 performs the determination of the imaging scene, the blood vessel identification, the lesion state, and the like based on one or two images that are required among the three images.

Similarly, the training data used for the learning of the prediction models of the imaging scene predictions 112, 114, 116, and the like may not include one or two of the vascular image, the blood flow waveform, and the envelope.

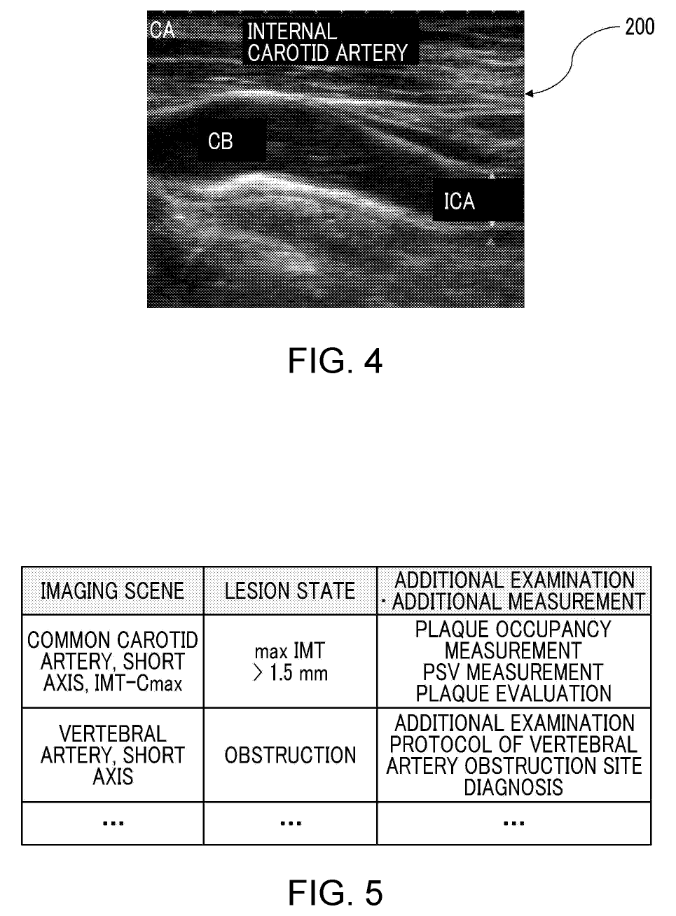

The display processing unit 16 may reflect the determination result of the blood vessel identification determination 140 on the vascular image 102 displayed on the screen. The example thereof is shown in FIG. 4.

FIG. 4 is a B-mode tomographic image showing a longitudinal cross section of the internal carotid artery. In this tomographic image, an abbreviation "ICA" indicating the internal carotid artery is displayed at the position of the internal carotid artery, and an abbreviation "CB" indicating the carotid sinus is displayed at the position of the carotid sinus.

In addition, the display processing unit 16 may reflect the determination result of the lesion state determination 160 on the screen being displayed. For example, in a case where the lesion state is determined to be the stenosis rate of 75%, the display processing unit 16 displays a message indicating the lesion state, such as “stenosis rate 75%”, at a predetermined position on the screen.

In addition, the display processing unit 16 may reflect the determination result of the imaging scene determination 120, for example, the scene name, on the screen being displayed.

Next, a detailed example of the additional examination suggestion processing 22 will be described. The additional examination suggestion processing 22 suggests an additional examination and/or measurement based on the imaging scene and the lesion state determined by the determination processing 100. This suggestion processing may be implemented by, for example, an LUT. FIG. 5 illustrates an LUT for the additional examination suggestion processing 22.

In the LUT of FIG. 5, information on the additional examination or measurement is registered in association with the combination of the imaging scene and the lesion state. For example, in an entry of the first row, in a case where the imaging scene is determined to be “common carotid artery, short axis IMT-Cmax” and the lesion state is determined to be “max IMT > 1.5 mm”, the additional examination or measurement is “plaque occupancy measurement”, “PSV measurement”, and “plaque evaluation”.

In addition, for example, in a case where the imaging scene is determined to be "vertebral artery, short axis" and the lesion state is determined to be "suspected obstruction" in the entry of the second row, the additional examination or measurement is "additional examination protocol for vertebral artery obstruction site diagnosis". The additional examination protocol is registered in advance in the ultrasound diagnostic system 1, and a series of examinations and measurements are defined in order. In this additional examination protocol, first, an ultrasound examination is performed in a pulse Doppler mode to obtain a blood flow waveform. Next, the presence or absence of the blood flow signal is confirmed from the blood flow waveform, and the presence or absence of the end-diastolic blood flow is confirmed in a case where the blood flow signal is present. Then, in a case where there is a diastolic end period blood flow, the average blood flow velocity is further measured, and in a case where the average blood flow velocity is equal to or greater than a predetermined threshold value, the ratio of the average blood flow velocities of the left and right sides is further measured.

The example shown in FIG. 5 is simple for the sake of description. In the column of the lesion state in the LUT, a combination of a plurality of lesion states may be registered. In addition, in the columns of the additional examination/additional measurement, a plurality of examinations or measurements or a plurality of examination protocols may be registered. The plurality of examinations or measurements, or the plurality of examination protocols may be ranked in descending order of the degree of conformity to the combination of the imaging scene and the lesion state.

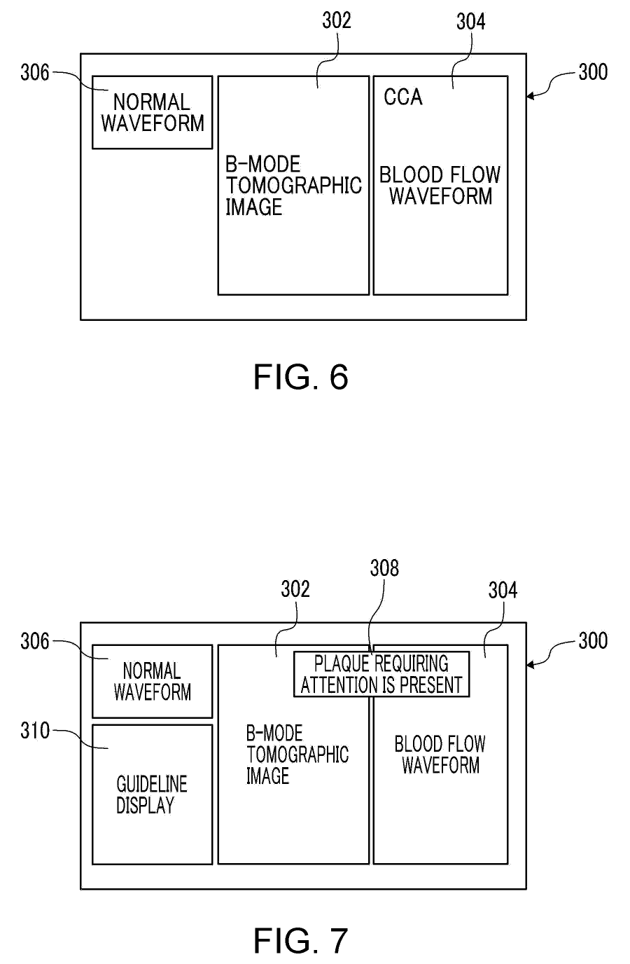

An example of a transition of the screen of the ultrasound diagnostic system 1 in a case where the additional examination and measurement suggestion function is used in the present embodiment will be described with reference to FIGS. 6 to 9.

FIG. 6 shows an example of a layout of a screen 300 in a case where an ultrasound examination is being performed. In this example, a B-mode tomographic image 302 and a blood flow waveform 304 obtained by the transmission and reception of the ultrasound beam by the probe 10 are displayed on the screen 300. In this example, the determination processing 100 determines that the imaging scene is a scene for a specific examination purpose of the common carotid artery, and "CCA" (CCA is an abbreviation for common carotid artery) indicating that the common carotid artery is being examined is displayed in the screen 300. The normal waveform 306 is an image of a typical example of a normal blood flow waveform for the imaging scene, and is used as a reference in the evaluation of the blood flow waveform 304 obtained from the subject under examination by the user at present. The normal waveform 306 is registered in the ultrasound diagnostic system 1 for each imaging scene. The example of the screen 300 is for the subject that is determined to be normal by the determination processing 100, and information on the lesion state or the like is not displayed.

FIG. 7 shows an example of a screen 300 in a case where the determination processing 100 determines that there is a lesion state. In this example, it is assumed that it is determined that there is a plaque that requires attention in the common carotid artery. In this case, an alert display 308 indicating that there is a plaque requiring attention is displayed on the screen 300, and a guideline display 310 indicating a guideline for diagnosing the plaque of the common carotid artery is displayed.

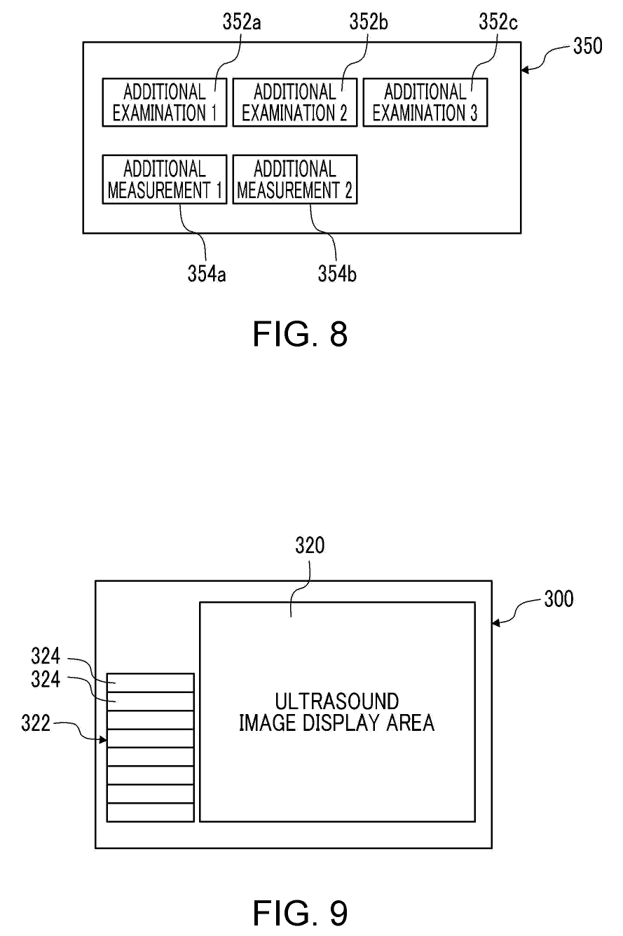

In addition, in this example, it is assumed that the ultrasound diagnostic system 1 includes a touch panel display that receives a touch input from the user, in addition to the main display device that displays the screen 300. In accordance with the determination of the lesion state, an additional examination suggestion screen 350 for suggesting the additional examination and the additional measurement corresponding to the combination of the current imaging scene and the lesion state is displayed on the touch panel display. FIG. 8 shows an example of the additional examination suggestion screen 350. In the example of FIG. 8, on the additional examination suggestion screen 350, several additional examination buttons 352a, 352b, and 352c and additional measurement buttons 354a and 354b corresponding to the combination of the imaging scene and the lesion state are displayed. Although not shown in the drawing in order to avoid complication, a character string indicating the name of the corresponding additional examination or additional measurement is displayed on each of the buttons 352a to 354b.

In a case where the user touches any of the buttons 352a to 354b with a finger, a user interface (UI) for the corresponding additional examination or additional measurement is displayed on the screen 300. For example, FIG. 9 illustrates a screen 300 displayed in a case where the additional examination button 352a is touched. Here, it is assumed that the additional examination button 352a represents a certain examination protocol. In this case, the UI displayed on the screen 300 in response to the pressing of the additional examination button 352a includes an examination protocol display field 322 in addition to the ultrasound image display area 320. In the examination protocol display field 322, a list in which the step display 324 representing each examination step constituting the examination protocol is arranged in the execution order of the steps is displayed. Execution is performed in order from the examination step indicated by the step display 324 at the beginning of the list. The step display 324 corresponding to the examination step being executed is highlighted so as to be distinguished from the other step displays. One or more ultrasound images obtained in the examination step currently being executed are displayed in the ultrasound image display area 320.

Returning to the description of FIG. 1, the finding suggestion processing 24 will be described. The finding suggestion processing 24 suggests a wording that is a finding candidate for the subject based on the combination of the imaging scene and the lesion state determined in the determination processing 100. The finding suggestion processing 24 is merely an example, but may be implemented by LUT. In the LUT, a wording that is a finding candidate is registered in association with a combination of an imaging scene and a lesion state.

In addition, the finding suggestion processing 24 may obtain the finding candidate in consideration of the results of the additional examination or the additional measurement executed according to the combination of the imaging scene and the lesion state. For example, in a case where the finding suggestion processing 24 is implemented by the LUT, the LUT registers a wording as a finding candidate in association with a combination of an imaging scene, a lesion state, and a result of an additional examination or an additional measurement.

A specific example will be described. For example, it is assumed that the determination processing 100 determines that the imaging scene is the common carotid artery long axis and determines that the lesion state is the end-diastolic blood flow velocity “high value” from the ultrasound image obtained in the examination currently being executed. In response to this determination result, the additional examination suggestion processing 22 suggests the comparison between the right and left sides of the common carotid artery end-diastolic blood flow velocity as the additional examination/measurement. The user executes the ultrasound examination of the right and left common carotid arteries in response to the suggestion. The information processing system 20 obtains the end-diastolic blood flow velocities of the right and left common carotid arteries from the data obtained by the ultrasound examination, and calculates a ratio between the end-diastolic blood flow velocities. Then, the information processing system 20 obtains the finding candidate from the value of the ratio. In this case, the finding candidate is "normal" in a case where the ratio is less than a predetermined first threshold value, "internal carotid artery distal obstruction lesion" in a case where the ratio is equal to or greater than the first threshold value and less than a second threshold value, and "obstruction due to cardiogenic embolism of internal carotid artery" in a case where the ratio is equal to or greater than the second threshold value. For example, in the LUT, “obstruction due to cardiogenic embolism of internal carotid artery” is registered as a finding candidate for a combination of an imaging scene “common carotid artery long axis”, a lesion state “end-diastolic blood flow velocity “high value””, and an additional examination/measurement result “left-right ratio of end-diastolic blood flow velocity of common carotid artery is equal to or greater than second threshold value”. The finding suggestion processing 24 obtains a finding candidate corresponding to a combination of the imaging scene and the lesion state obtained in the determination processing 100 and the result of the additional examination and measurement from the LUT.

It should be noted that the implementation of the finding suggestion processing 24 using the LUT is merely an example. The finding suggestion processing 24 can be programmed in various ways. In addition, the finding suggestion processing 24 may be implemented by a machine learning model.

The finding suggestion processing 24 displays a wording that is a candidate for the obtained finding on the screen, and requests confirmation and correction from the user. In a case where the displayed candidate wording is appropriate, the user presses, for example, an "OK" button displayed on the screen. In addition, in a case where the candidate wording needs to be corrected, for example, the user corrects the wording by operating a keyboard or the like and then presses the "OK" button. As a result, the findings are completed and registered in the patient database.

Next, the report draft creation processing 26 will be described. The report draft creation processing 26 creates a draft of a report on the examination result of the subject based on the combination of the imaging scene and the lesion state determined in the determination processing 100. For example, a template of a report of the examination result is determined according to the imaging scene.

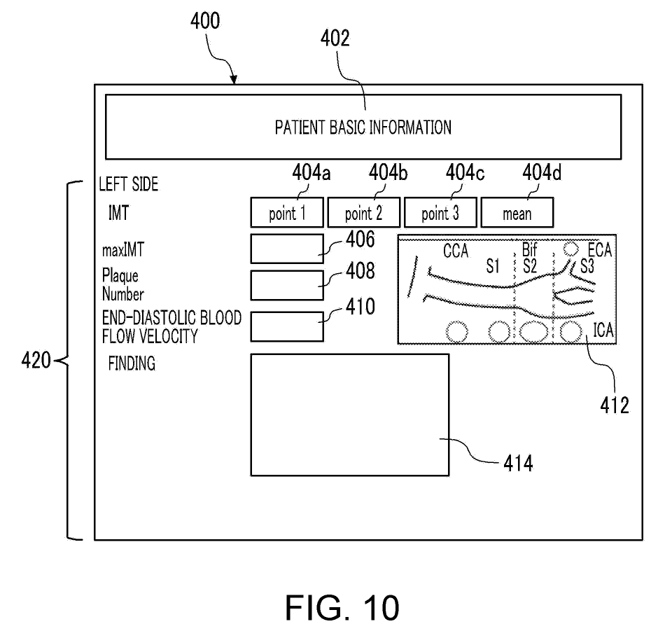

FIG. 10 illustrates a template 400 of a report corresponding to the imaging scene “common carotid artery long axis”. The template 400 includes first the patient basic information 402 and the examination result field 420. The patient basic information 402 includes information for specifying the patient, such as an ID, a name, a gender, an age, a main complaint, a past history, and an examination date and time of the patient, and information indicating a background of the examination. In the examination result field 420, various measurement values, a schema 412, and a finding 414 obtained in the examination are shown. The measurement values include IMT 404a, 404b, and 404c at three predetermined sites, an average value 404d of IMTs, a maximum IMT (max IMT) 406, the number of plaques 408, and an end-diastolic blood flow velocity 410. The schema 412 shows a schematic diagram showing a configuration of blood vessels in an observation range in the imaging scene. In a case where an abnormal finding such as a plaque is detected, a site related to the abnormal finding is displayed in the schematic diagram in the schema 412. The finding 414 is a finding of the examiner or the like for the examination, and is created based on, for example, the finding candidate suggested by the finding suggestion processing 24.

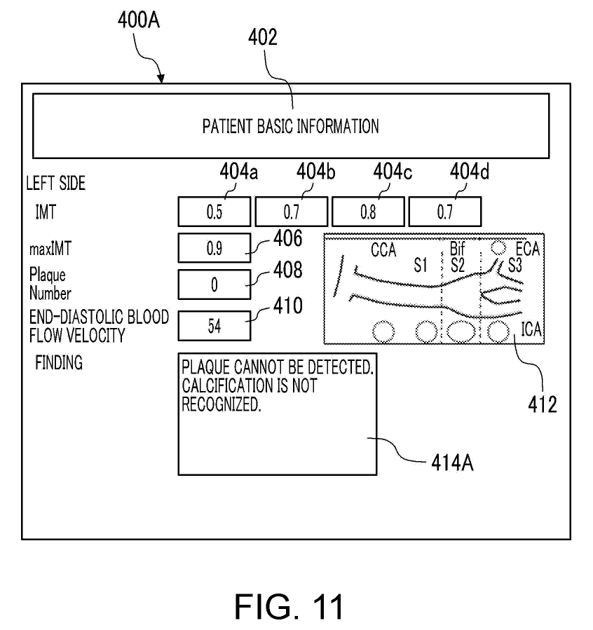

FIG. 11 illustrates a report 402A in a case where no abnormal findings are recognized in the examination of the “common carotid artery long axis”. In this example, the wording indicating that the plaque and the calcification, which are the detection targets, are not present is shown in the finding 414A.

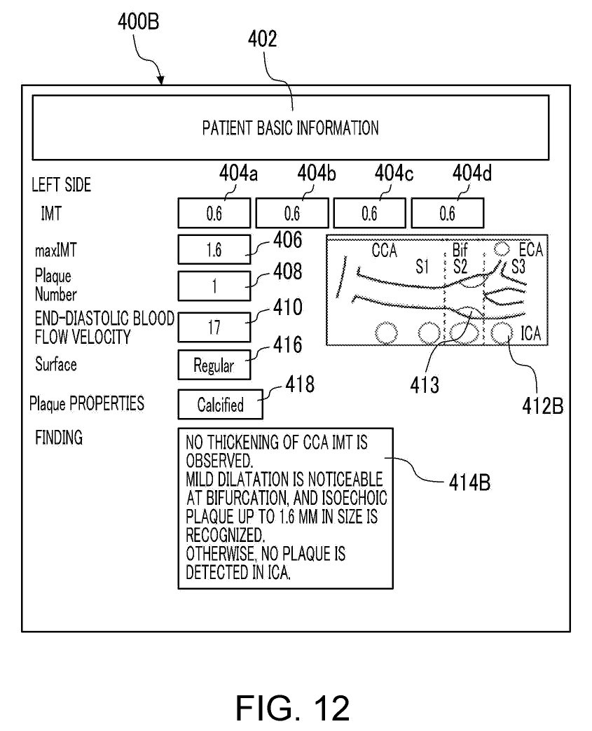

FIG. 12 illustrates a report 402B in a case where an abnormal finding related to the plaque is recognized in the examination of the "common carotid artery long axis". In this example, the schema 412B shows that the plaque 413 is present in the vascular branch part. In addition, the findings 414B indicate information on the plaque detected from the vascular branch, information on the presence or absence of the plaque in another vascular site, and the like.

In the report draft creation processing 26, a template of a report corresponding to the imaging scene determined in the determination processing 100 is read out, and the measurement value obtained in the examination is input to the template. In addition, in a case where the finding indicating the predetermined abnormality, such as the plaque, is generated by the finding suggestion processing 24, the report draft creation processing 26 adds a figure indicating a site corresponding to the finding to the schema 412. In addition, in the report draft creation processing 26, the wording of the finding candidate generated by the finding suggestion processing 24 is input to the field of the finding 414. By such processing, the drafts of the reports 400A and 400B are generated. The user checks the content of the report draft, makes a correction as necessary, and confirms the content of the final report. The confirmed report is registered in the patient database.

Returning to the description of FIG. 1, the approach determination processing 28 will be described. The implementation of the approach determination processing 28 can be performed using, for example, the prediction models of the blood vessel identification predictions 132, 134, and 136. In this example, the blood vessel identification predictions 132, 134, and 136 are trained to learn the determination of the appropriateness of the approach of the probe 10. For this purpose, the blood vessel identification predictions 132, 134, and 136 are trained using training data including information on whether or not the approach of the probe 10 is appropriate in the labeled data. The learned blood vessel identification predictions 132, 134, and 136 output information indicating the appropriateness of the approach of the probe 10 in addition to the blood vessel name for the input vascular image 102, blood flow waveform 104, and envelope 106. The information on the appropriateness of the approach output by the blood vessel identification predictions 132, 134, and 136 is integrated by the blood vessel identification determination 140, and the approach determination processing 28 is performed based on the integration result. As a result, whether or not the approach of the probe 10 is appropriate for the ultrasound image currently being displayed is displayed on the screen. The user can adjust the position, angle, and the like at which the probe 10 is applied by referring to this display.

In the present embodiment, each processing is executed by any computer. In addition, any computer may execute these types of processing by a processor as hardware, a program as software, or a combination thereof. In such a case, the processor is configured to execute various types of processing in the present embodiment in cooperation with the program, and may function as each unit or each means in the present embodiment. In addition, the execution order of the processing by the processor is not limited to the above-described order and may be changed as appropriate. Any computer may be a general-purpose computer, a computer for a specific application, a workstation, or another system capable of executing each process.

The processor may be composed of one or a plurality of pieces of hardware, and types of hardware are not limited. For example, the processor may be configured by a programmable logic device such as a central processing unit (CPU), a micro processing unit (MPU), or a field programmable gate array (FPGA), a dedicated circuit for executing specific processing, such as an application-specific integrated circuit (ASIC), or hardware such as a graphic processing unit (GPU) or a neural processing unit (NPU). Moreover, the type of hardware may be a combination of different kinds of hardware. In a case where the plurality of types of hardware are configured to execute one or a plurality of types of processing of a certain processor, the plurality of types of hardware may exist in devices physically separated from each other or may exist in the same device. Furthermore, in any of the embodiments, the order of each processing performed by the processor is not limited to the above-described order, and may be changed as appropriate. In addition, hardware is implemented in a form of an electric circuit (circuitry) in which circuit elements, such as semiconductor elements, are combined.

Further, the program may be software, such as firmware or a microcode. Furthermore, the program may be, for example, a program module group, and each function thereof may be implemented by a processor configured to execute each function. The program may be a program code or a plurality of code segments stored in one or a plurality of non-transitory computer-readable media (for example, a storage medium and other storages). The program may be stored in the plurality of non-transitory computer-readable media existing in physically separated devices. The program code or the code segment may represent any combination of procedures, functions, subprograms, routines, subroutines, modules, software packages, classes, instructions, data structures, or program statements. The program code or the code segment may be connected to another code segment or a hardware circuit by transmitting and receiving information, data, arguments, parameters, or contents in the memory.

What is claimed is:

1. An ultrasound diagnostic system comprising: a processor,
wherein the processor is configured to:
execute, from at least one of a vascular image, a blood flow waveform, or an envelope of the blood flow waveform, obtained by transmission and reception of ultrasonic waves to and from a subject in an ultrasound examination step being executed, determination processing of determining an imaging scene and a lesion state of the ultrasound examination step being executed; and
output, based on a combination of the determined imaging scene and lesion state, suggestion information for suggesting additional ultrasound examination or measurement for the ultrasound examination step being executed.

2. The ultrasound diagnostic system according to claim 1,
wherein the processor is configured to determine the imaging scene and the lesion state in the ultrasound examination step being executed, by using a machine learning model trained to output, in a case where an input of at least one of the vascular image, the blood flow waveform, or an image of the envelope is received, the imaging scene and the lesion state corresponding to the input.

3. The ultrasound diagnostic system according to claim 1,
wherein the processor is further configured to present a finding candidate for the subject, based on a result obtained by executing an ultrasound examination or measurement specified by a user among the additional ultrasound examination or measurement shown in the suggestion information and on the imaging scene and the lesion state determined by the determination processing.

4. The ultrasound diagnostic system according to claim 3,
wherein the processor is further configured to create a report document including a finding created based on the finding candidate.

5. The ultrasound diagnostic system according to claim 2,
wherein the processor is further configured to present a finding candidate for the subject, based on a result obtained by executing an ultrasound examination or measurement specified by a user among the additional ultrasound examination or measurement shown in the suggestion information and on the imaging scene and the lesion state determined by the determination processing.

6. The ultrasound diagnostic system according to claim 5,
wherein the processor is further configured to create a report document including a finding created based on the finding candidate.

7. The ultrasound diagnostic system according to claim 1,
wherein the processor is further configured to execute determination of appropriateness for an approach of an ultrasound probe from at least one of the vascular image, the blood flow waveform, or an image of the envelope, and
the determination of the appropriateness for the approach is performed using a second machine learning model trained to output, in a case where an input of the at least one of the vascular image, the blood flow waveform, or the image of the envelope is received, the appropriateness of the approach corresponding to the input.

8. A non-transitory computer readable medium storing a program causing a computer to execute a process comprising:
executing, from at least one of a vascular image, a blood flow waveform, or an envelope of the blood flow waveform, obtained by transmission and reception of ultrasonic waves to and from a subject in an ultrasound examination step being executed, determination processing of determining an imaging scene and a lesion state of the ultrasound examination step being executed; and
outputting, based on a combination of the determined imaging scene and lesion state, suggestion information for suggesting additional ultrasound examination or measurement for the ultrasound examination step being executed.

9. A method for ultrasound diagnostic processing, comprising:
executing, from at least one of a vascular image, a blood flow waveform, or an envelope of the blood flow waveform, obtained by transmission and reception of ultrasonic waves to and from a subject in an ultrasound examination step being executed, determination processing of determining an imaging scene and a lesion state of the ultrasound examination step being executed; and
outputting, based on a combination of the determined imaging scene and lesion state, suggestion information for suggesting additional ultrasound examination or measurement for the ultrasound examination step being executed.
